(19) United States
(12) Patent Application Publication
(10) Pub. No.: US 2016/0333387 A1
(43) Pub. Date: Nov. 17, 2016

(54) Monitoring Microbial Growth Rates

(71) Applicant: Savannah River Nuclear Solutions, LLC, Aiken, SC (US)

(72) Inventors: Charles E. Turick, Aiken, SC (US); Charles E. Milliken, Evans, GA (US); Hector Colon-Mercado, Aiken, SC (US); Scott D. Greenway, Aiken, SC (US)

(21) Appl. No.: 14/710,215

(22) Filed: May 12, 2015

(51) Int. Cl. C12Q 1/02; G01N 27/02

(52) U.S. Cl. CPC C12Q 1/02; G01N 27/026

(57) ABSTRACT

Systems and methods of monitoring microbial growth rates are provided. In particular, a sensing electrode having a working electrode and a counter electrode can be positioned in a microbial environment. An alternating current signal can be applied to the working electrode. The signal can then propagate through the microbial environment and can be measured at the counter electrode. The presence of microorganisms in the microbial environment can cause changes in the signal as it propagates through the microbial environment. Such changes in the signal can be used to determine one or more signal parameters associated with the microbial environment. The one or more signal parameters can be used to determine a microbial growth rate. Nutrient concentrations can then be adjusted in the microbial environment to facilitate an optimal growth rate.

### GOVERNMENT SUPPORT CLAUSE

This invention was made with Government support under Contract No. DE-AC09-08SR22470, awarded by the U.S. Department of Energy. The Government has certain rights in the invention.

### FIELD

The present disclosure relates generally to monitoring cellular activity and more particularly to monitoring cellular activity of microorganisms.

### BACKGROUND

Monitoring microbial activity can be important for many applications, including bioremediation, waste containment, and other applications. For instance, bioremediation is a waste management technique that involves the use of organisms to remove or neutralize pollutants from a contaminated site. Bioremediation can involve microorganisms enzymatically attacking the pollutants and converting them to harmless products. In particular, a type of bioremediation called biostimulation can involve the manipulation of environmental parameters to stimulate microorganisms capable of bioremediation and to facilitate microbial growth and degradation.

Electrochemical impedance spectroscopy (EIS) is a technique that can be used to monitor various parameters of electrochemical systems through monitoring of impedance parameters. EIS systems can include a working electrode and a counter electrode. The EIS system can evaluate the impedance of a medium by applying an AC signal with variable frequency through the pair of electrodes while measuring the resulting current. The real and imaginary parts of the impedance can be plotted as a function of frequency and analyzed to extract parameters of the system.

### SUMMARY

Aspects and advantages of embodiments of the present disclosure will be set forth in part in the following description, or may be learned from the description, or may be learned through practice of the embodiments.

One example aspect of the present disclosure is directed to a system for monitoring cellular activity. The system includes a sensing electrode submerged in a microbial environment. The sensing electrode has a working electrode and a counter electrode. The system further includes a signal source coupled to the sensing electrode. The signal source is configured to apply an alternating current signal to the working electrode at one or more frequencies. The system further includes a processing device configured to perform operations. The operations include detecting a measured signal at the counter electrode. The operations further include determining an imaginary conductivity associated with the measured signal. The operations further include correlating the imaginary conductivity of the measured signal to an oxygen utilization rate associated with the microbial environment.

Other example aspects of the present disclosure are directed to systems, methods, apparatus, tangible, non-transitory computer-readable media, user interfaces, memory devices, and electronic devices for monitoring cellular activity.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 depicts an example system for monitoring cellular activity according to example embodiments of the present disclosure;

FIG. 2 depicts an example sensing electrode according to example embodiments of the present disclosure;

FIG. 3 depicts a flow diagram of an example method of monitoring cellular activity according to example embodiments of the present disclosure;

FIG. 4 depicts a diagram of imaginary conductivity of a microbial environment over a range of frequencies according to example embodiments of the present disclosure;

FIG. 5 depicts a diagram of imaginary conductivity and oxygen utilization of a microbial environment according to example embodiments of the present disclosure; and

FIG. 6 depicts a diagram of example microbial growth according to example embodiments of the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Example aspects of the present disclosure are directed to monitoring cellular activity in a microbial environment. For instance, a sensing electrode can be embedded in a subsurface probe. The sensing electrode can include a working electrode and a counter electrode. The subsurface probe can be positioned in a microbial environment. As used herein, a microbial environment can be any environment or media that acts as a habitat for microorganisms. For instance, the microbial environment can be a subsurface of the Earth (e.g. groundwater, soil, etc.), or a controlled environment such as a bioreactor. The sensing electrode can be used to determine characteristics of microorganisms through the implementation of electrochemical impedance spectroscopy (EIS) in the microbial environment. As indicated above, EIS can be used to determine impedance parameters, which can correspond to characteristics of microorganisms in the microbial environment.

In example embodiments, when the subsurface probe and the embedded sensing electrode are positioned in a microbial environment, an alternating current (AC) signal can be applied to the working electrode. In particular, the signal can be an AC potential applied at the working electrode over a range of frequencies. Microorganisms located in the microbial environment can cause the signal to change as the signal propagates through the microbial environment. The signal can then be measured at the counter electrode. In particular, an AC current can be measured at the counter electrode. The applied potential and the measured current can then be used to determine one or more signal parameters associated with the microbial environment.

Such signal parameters can be derived at least in part from changes in the applied signal due to the presence of microorganisms in the microbial environment. For instance, such changes can include changes in the current, phase, and/or amplitude of the signal. Such changes can vary over the range of frequencies. The identified signal parameters can include impedance parameters. The impedance parameters can include absolute impedance, real (e.g. in-phase) impedance, and/or imaginary (out-of-phase) impedance associated with the signal. Such impedance parameters can be used to further determine conductivity parameters (e.g. real and/or imaginary conductivity) associated with the microbial environment.

The various signal parameters can provide information relating to microbial activity in the microbial environment. Such information can be useful regarding microbial cultures. In particular, the signal parameters can change as the microbial cultures in the microbial environment convert carbon sources to waste products. Such changes can be used to identify microbial activity. In example embodiments, absolute impedance can be plotted against frequency (e.g. Bode plot) to determine general information associated with impedance and admittance. Further, imaginary impedance can be plotted against real impedance (e.g. Nyquist plot) to determine information indicative of reaction rates and diffusion phenomena. As another example, phase shift of the measured current relative to the applied potential can correspond to geochemical frequencies at low frequencies (e.g. about 0.01 Hz to about 1.0 Hz). Further, at mid-level frequencies (e.g. about 10 Hz to about 1000 Hz), phase shift can correspond to microbial density.

In example embodiments, information relating to microbial growth can be determined from the conductivity of the microbial environment. For instance, imaginary conductivity can correspond to the ability to store energy and/or lipid membrane signatures. Further, imaginary conductivity can correspond to oxygen utilization rate, substrate utilization rate, terminal electron acceptor (TEA) utilization rate, and cellular activity (e.g. energy flow). This information can be used to determine a microbial growth rate associated with the microbial environment.

As used herein, microbial growth can be defined as the asexual reproduction (e.g. cell division) of a microorganism into two daughter cells. In flow through environments (e.g. groundwater, bioreactors, etc.), microbial growth is a function of the rate of nutrient addition into the environment. In particular, microbial growth rates can correspond to the concentration of limiting nutrients (e.g. carbon sources) available to the microorganisms in the microbial environment. Such limiting nutrients can provide a source of organic carbon and energy to the microorganism cells. In particular, microbial growth rate can correspond to the rate of carbon source utilization and electron transfer to the TEA.

Growth can further correspond to the flow rate of water and nutrients through the environment. Steady state growth conditions can occur when microbial growth, nutrient additions and dilution are balanced in the environment. At steady state growth conditions, the microbial growth rate approaches a maximum growth rate. To maintain steady state conditions, nutrient concentrations can be varied with changes in microbial growth rates due to physiological state (e.g. temperature changes, growth stage, etc.), microbial concentration, and flow rate.

Accordingly, once the microbial growth rate is determined, nutrient concentrations can be adjusted based at least in part on the determined growth rate. For instance, if the growth rate falls below a threshold, the nutrient concentration can be increased to increase growth rate. In example embodiments, the nutrient concentration can be increased by increasing an oxygen utilization rate and/or increasing a carbon source utilization rate associated with the microbial environment. The adjustment of nutrient concentrations can be used to facilitate steady state conditions, and an optimal microbial growth rate.

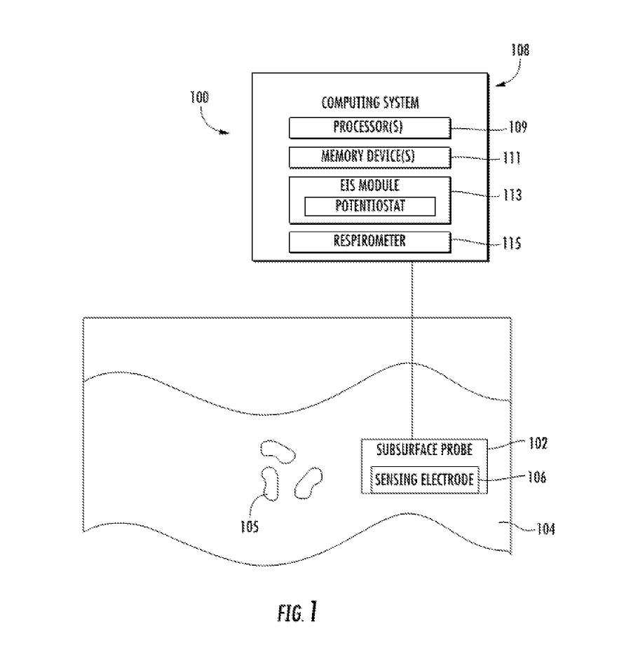

Referring now to the figures, FIG. 1 depicts an example system 100 for monitoring cellular activity in a microbial environment according to example embodiments of the present disclosure. Microbial environment System 100 can include a subsurface probe 102 positioned in a microbial environment 104. Microbial environment 104 can include microorganisms 105. Subsurface probe can include a sensing electrode 106. Sensing electrode 106 can include a working electrode and a counter electrode, and can be used to monitor microbial growth in microbial environment 104 through EIS.

When positioned in microbial environment 104, sensing electrode 106 can implement EIS to determine one or more signal parameters (e.g. impedance parameters, conductivity parameters, etc.) associated with microbial environment 104. The signal parameters can be determined, for instance, by a computing system 108 coupled to sensing electrode 106 based on signals detected at sensing electrode 106.

Computing system 108 can be any suitable type of computing device, such as a general purpose computer, special purpose computer, laptop, desktop, mobile device, smartphone, tablet, wearable computing device, a display with one or more processors, or other suitable computing device. Computing system 108 can include one or more processor(s) 109 and one or more memory device(s) 111.

The one or more processor(s) 109 can include any suitable processing device, such as a microprocessor, microcontroller, integrated circuit, logic device, one or more central processing units (CPUs), graphics processing units (GPUs) dedicated to efficiently rendering images or performing other specialized calculations, and/or other processing devices. The one or more memory device(s) 111 can include one or more computer-readable media, including, but not limited to, non-transitory computer-readable media, RAM, ROM, hard drives, flash memory, or other memory devices.

The one or more memory device(s) 111 store information accessible by the one or more processor(s) 109, including instructions that can be executed by the one or more processors. For instance, the memory devices can store instructions for monitoring cellular activity in a microbial environment according to example embodiments of the present disclosure.

Computing system 108 can further include a respirometer 115 used to measure a rate of respiration of microorganisms 105, and an EIS module 113. EIS module 113 can be used to determine impedance parameters associated with microbial environment 104. EIS module can include a potentiostat used to provide a signal to sensing electrode 106 and detect a measured signal at sensing electrode 106.

As used herein, the term “module” can be defined as computer logic used to provide desired functionality. As such, a module can be implemented in various manners. For instance, a module can be implemented in hardware devices, application specific circuits, firmware and/or software used to control one or more general purpose processors. In example embodiments, modules can be program code files that are stored on a storage device, loaded into memory and executed by a processor. In alternative embodiments, modules can be provided from computer program products (e.g. computer executable instructions) that are stored in a tangible computer-readable storage medium such as RAM, a hard disk or optical or magnetic media.

According to example embodiments of the present disclosure, an AC potential can be applied at the working electrode of sensing electrode 106. The signal can propagate through the microbial environment, and can then be received by the counter electrode. An AC current can then be measured at the counter electrode, and data indicative of the measured current can then be received by computing system 108. Computing system 108 can then determine signal parameters associated with surface 104 based at least in part on the measured current and the applied potential. As described above, the signal parameters can correspond to characteristics of microorganisms on surface 104.

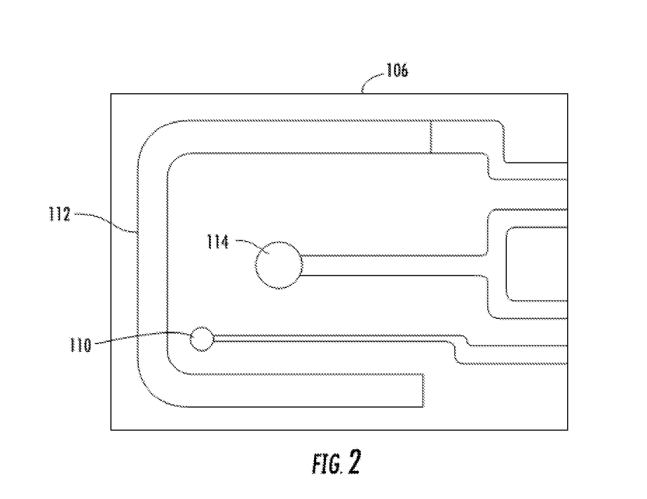

FIG. 2 depicts an example sensing electrode 106 according to example embodiments of the present disclosure. Although FIG. 2 depicts a three-electrode cell, it will be appreciated that various other electrode configurations can be used, such as a two electrode cell or a four electrode cell. Sensing electrode 106 can be a flat, patterned electrode, and can include a reference electrode 110, a counter electrode 112 and a working electrode 114. During EIS, reference electrode 110 can have a constant (or near constant), known reference potential. In this manner, a potential applied at working electrode 114 can be measured relative to the reference potential. The signal applied at working electrode 114 can propagate through a medium and can be received by counter electrode 112. According to example embodiments of the present disclosure, when sensing electrode 106 is positioned in a microbial environment, microorganisms located in the microbial environment can cause alterations in the signal. As indicated above, the changes in the signal can correspond to signal parameters associated with the microbial environment, which can correspond to various microbial characteristics of the microbial environment.

In alternative embodiments, sensing electrode 106 can be used to implement various other electrochemical techniques such as voltammetry. For instance, linear sweep voltammetry and/or cyclic voltammetry can be implemented using sensing electrode 106. In linear sweep voltammetry, the AC potential applied at the working electrode is increased linearly over time. In cyclic voltammetry, the AC potential applied at the working electrode is cycled over time. According to example embodiments of the present disclosure, linear sweep voltammetry and cyclical voltammetry can be used to more fully interrogate the microbial environment for such features as contaminant concentrations and/or electron shuttles, with some potential to detect unknown or unexpected contaminants.

Sensing electrode 106 can be made from various suitable materials. For instance, sensing electrode 106 can include a ceramic substrate. Working electrode 114 and counter electrode 112 can be gold or platinum electrodes. Reference electrode 110 can be a silver and/or silver chloride electrode. It will be appreciated by those skilled in the art that various other suitable materials can be used.

FIG. 3 depicts a flow diagram of an example method (200) of monitoring cellular activity according to example embodiments of the present disclosure. FIG. 3 can be implemented using a sensing electrode, such as sensing electrode 106 of FIG. 2, and one or more computing devices, such as computing system 108 of FIG. 1. In addition, FIG. 3 depicts steps performed in a particular order for purposes of illustration and discussion. Those of ordinary skill in the art, using the disclosures provided herein, will understand that the steps of any of the methods disclosed herein can be adapted, omitted, rearranged, expanded, or modified in various ways without deviating from the scope of the present disclosure.

At (202), method (200) can include positioning a sensing electrode in a microbial environment. The sensing electrode can have a working electrode and a counter electrode, and can be used to implement EIS and/or various other electrochemical techniques in the microbial environment.

At (204), method (200) can include applying an AC signal at the working electrode. For instance, the applied signal can be an AC potential. At (206), method (200) can include detecting a measured signal at the counter electrode. For instance, the measured signal can be a current signal. As indicated above, as the applied AC signal propagates through the microbial environment, the presence of microorganisms can cause alterations in the signal.

At (208), method (200) can include determining one or more characteristics of the measured signal. For instance, the one or more characteristics of the measured signal can include a phase change of the signal relative to the applied signal, and/or various impedance parameters associated with the surface and the aqueous medium (e.g. imaginary impedance, real impedance, absolute impedance, conductivity, relative permittivity, etc.). Impedance is a measurement of resistance in the presence of an AC potential. Impedance can be determined by applying an AC potential and measuring the current through a cell. In complex systems, an impedance value (Z) can include a real (e.g. in-phase) impedance (Z′) and an imaginary (e.g. out-of-phase) impedance (Z″). Imaginary impedance can be derived from a phase shift of the current relative to the AC potential. The real and imaginary impedances can be used to derive an absolute (e.g. absolute magnitude) impedance value (|Z|). In particular, absolute impedance can correspond at least in part to the degree to which a medium changes the amplitude and/or phase of a signal propagating through the medium. Absolute impedance can be calculated as follows:

|Z|=\sqrt{Z′²+Z″²}

The one or more characteristics of the measured signal can further include conductivity parameters associated with the microbial environment. Conductivity measures the ability of a material to conduct an electric current. Conductivity is the reciprocal of electrical resistivity, and can be derived from resistivity. In complex systems, conductivity can be derived from impedance, and can have a real component and an imaginary component. Real conductivity (σ′) can be calculated as follows:

$${σ}^{′} = \frac{- {Z}^{n}}{{Z}^{′2} + {Z}^{n 2}} \cdot \frac{d}{π {R}^{2}}$$

where d is the distance between the working and counter electrodes, and R is the resistance of an ionic solution. Imaginary conductivity (σ″) can be calculated as follows:

σ″=ε′ε₀ω

where ε′ is real permittivity, ε₀ is the permittivity of free space (8.854×10⁻¹⁴ F/cm), and ω is the angular frequency.

At (210), method (200) can include analyzing the one or more characteristics of the measured signal to determine characteristics of microorganisms in the microbial environment. In example embodiments, such characteristics of microorganisms can include an oxygen utilization rate, which can correspond to microbial growth. In alternative embodiments, the characteristics of microorganisms can further include energy storage, microbial activity, mineralogy, reaction rates, biogeochemical changes, polarization, cell viability etc.

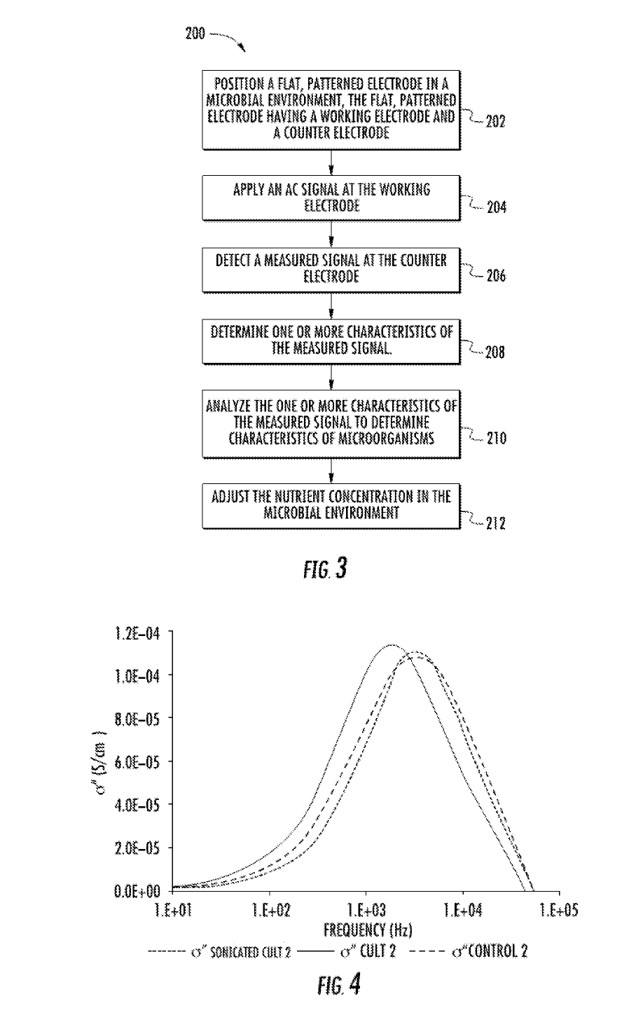

For instance, FIG. 4 depicts imaginary conductivity associated with various surfaces over a range of frequencies. In FIG. 4, Cult 2 represents an aluminum coupon with an attached microbial biofilm having high biomass concentrations, Sonicated Cult 2 represents an aluminum coupon after having a biofilm removed through sonication, and Control represents a sterile aluminum coupon. As depicted, Cult 2 demonstrates differences in imaginary conductivity when compared to Sonicated Cult 2 and Control. Such differences in imaginary conductivity can be used to measure changes in microbial characteristics of the aluminum coupons. In particular, changes in imaginary conductivity can correspond to energy storage and/or polarization related to cell activity on a surface.

Imaginary Conductivity can further correspond to oxygen utilization of microorganisms. For instance, FIG. 5 depicts imaginary conductivity and oxygen utilization rate over time. The oxygen utilization rate can be determined, for instance, using respirometer 115 of FIG. 1. The oxygen utilization rate of microorganisms in a microbial environment can be indicative of microbial growth. In particular, a microorganism can gain energy from consuming an organic carbon source. Further, a microorganism can gain energy from converting oxygen into water (e.g. oxygen utilization). The amount of carbon consumed per unit time is proportional to both gained energy and the amount of oxygen required by the microorganism. Once the energetic maintenance needs of the microorganism are met, excess energy and organic carbon can be used in microbial growth.

At (212), method (200) can include adjusting the nutrient concentration in the microbial environment based at least in part on the determined microbial characteristics. For instance, the nutrient concentration can be adjusted based at least in part on imaginary conductivity. The nutrient concentrations can be adjusted to achieve a maximum, or near maximum, microbial growth rate. As indicated above, the microbial growth rate can approach maximum in steady state conditions. Accordingly, the nutrient concentration can be adjusted to maintain steady state conditions. For instance, if the microbial growth rate falls below maximum growth rate, the nutrient concentration in the microbial environment can be increased (e.g. by adding nutrients). In alternative embodiments, nutrient concentrations can be further adjusted based at least in part on microbial concentration and/or flow rate.

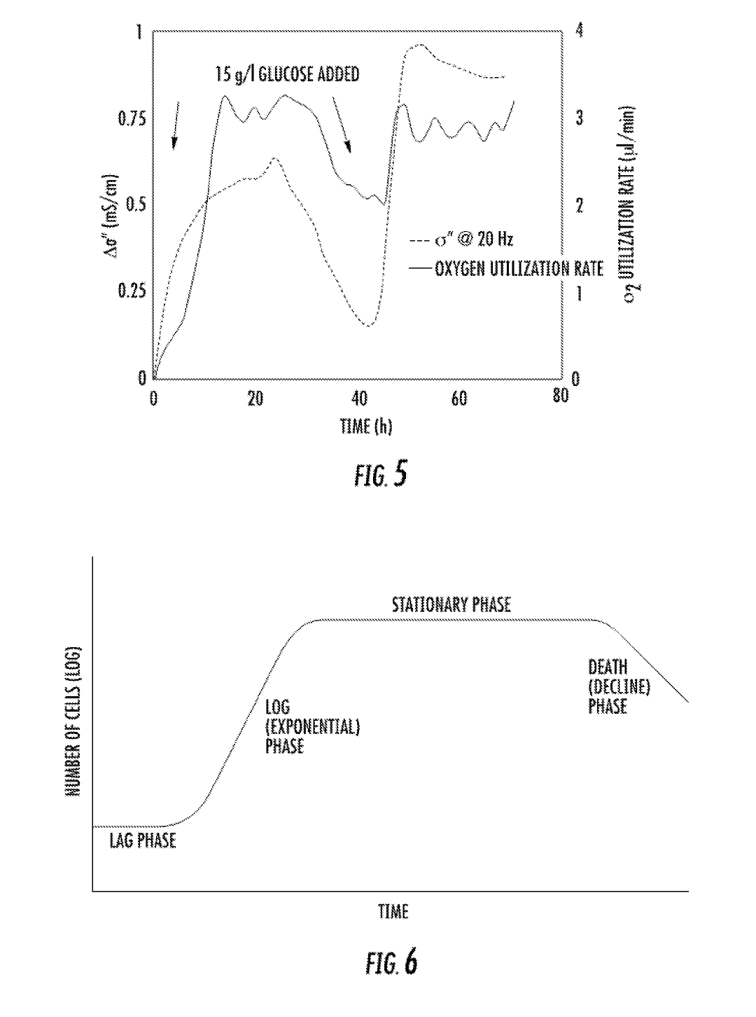

For instance, FIG. 6 depicts a diagram of microbial growth over time. As shown, microbial growth can include four phases. In the lag phase, microorganisms are generally not yet able to divide. Accordingly, little to no growth occurs in the lag phase. The exponential phase is a period of optimal microbial growth. In the exponential phase, the number of new microorganisms appearing per unit time is proportional to the present population. Exponential growth cannot typically continue indefinitely as the microbial environment may run out of nutrients required for microbial growth.

Accordingly, the stationary phase occurs when growth slows or stops. In the stationary phase, the growth rate and the death rate of microorganisms in the microbial environment may be equal. The final phase is the death phase when microorganisms begin to die off. This can occur due to a lack of nutrients, or other various conditions in the microbial environment (e.g. high temperature).

As described above, the microbial growth rate in a microbial environment can be determined based at least in part on signal parameters (e.g. imaginary conductivity) associated with the microbial environment. Once the growth rate is determined, nutrient concentrations in the microbial environment can be adjusted as desired to adjust the growth rate. For instance, if a growth rate has reached the death phase, nutrient concentrations in the microbial environment can be increased to increase the growth rate such that microbial growth is in the exponential phase. In alternative embodiments, nutrient concentrations can be decreased in order to decrease microbial growth as desired.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

1. A system for monitoring cellular activity, the system comprising:
a sensing electrode submerged in a microbial environment, the sensing electrode having a working electrode and a counter electrode;
a signal source coupled to the sensing electrode, the signal source configured to apply an alternating current signal to the working electrode at one or more frequencies; and
a processing device configured to perform operations, the operations comprising:
detecting a measured signal at the counter electrode;
determining an imaginary conductivity associated with the measured signal; and
correlating the imaginary conductivity to an oxygen utilization rate associated with the microbial environment.

2. The system of claim 1, wherein the one or more operations further comprise adjusting a concentration of nutrients in the microbial environment based at least in part on the oxygen utilization rate.

3. The system of claim 1, wherein the oxygen utilization rate is indicative of microbial growth rate in the microbial environment.

4. The system of claim 2, wherein the nutrient concentration is further adjusted based at least in part on at least one of a microbial concentration and flow rate in the microbial environment.

5. The system of claim 2, wherein adjusting the concentration of nutrients comprises increasing the concentration of nutrients to increase microbial growth rate in the microbial environment.

6. The system of claim 1, wherein the measured signal at the counter electrode comprises a current signal.

7. The system of claim 1, wherein the imaginary conductivity associated with the measured signal is determined at least in part from a phase shift of the measured signal relative to the applied signal.

8. The system of claim 1, wherein the microbial environment is groundwater in a subsurface of the Earth.

9. The system of claim 1, wherein the microbial environment is a bioreactor.

10. The system of claim 1, sensing electrode is a flat, patterned electrode.

11. A method of monitoring cellular activity, the method comprising:
positioning a sensing electrode in a microbial environment, the sensing electrode comprising a counter electrode and a working electrode;
applying an alternating current signal at the working electrode;
detecting a measured signal at the counter electrode; and
determining an imaginary conductivity associated with the measured signal;
correlating the imaginary conductivity to an oxygen utilization rate associated with the microbial environment.

12. The method of claim 11, further comprising adjusting a concentration of nutrients in the microbial environment based at least in part on the oxygen utilization rate.

13. The method of claim 12, wherein the oxygen utilization rate is indicative of microbial growth rate in the microbial environment.

14. The method of claim 13, further comprising further adjusting the concentration of nutrients based at least in part on at least one of a microbial concentration and flow rate associated with the microbial environment.

15. The method of claim 13, wherein adjusting the concentration of nutrients comprises increasing the concentration of nutrients to increase a microbial growth rate in the microbial environment.

16. A system for controlling microbial growth rate, the system comprising:
a flat, patterned electrode submerged in a microbial environment, the flat, patterned electrode having a working electrode and a counter electrode;
a signal source coupled to the flat, patterned electrode, the signal source configured to apply an alternating current signal to the working electrode at one or more frequencies; and
a processing device configured to perform operations, the operations comprising:
applying an alternating current signal at the working electrode;
detecting a measured current signal at the counter electrode;
determining an imaginary conductivity associated with the microbial environment and the measured current signal; and
increasing a concentration of nutrients in the microbial environment based at least in part on the imaginary conductivity;
wherein the imaginary conductivity is indicative of an oxygen utilization rate in the microbial environment.

17. The system of claim 16, wherein the oxygen utilization rate is indicative of microbial growth rate in the microbial environment.

18. The system of claim 16, wherein the imaginary conductivity is derived at least in part from a determined phase shift of the measured signal relative to the applied signal.

19. The system of claim 16, wherein the processing device is further configured to further increase the nutrient concentration based at least in part on at least one of a microbial concentration and flow rate in the microbial environment.

20. A method of monitoring cellular activity, the method comprising:
positioning a sensing electrode in a microbial environment, the sensing electrode comprising a counter electrode and a working electrode;
applying an alternating current signal at the working electrode;
detecting a measured signal at the counter electrode; and
determining an imaginary conductivity associated with the measured signal;
correlating the imaginary conductivity to a terminal electron acceptor utilization rate associated with the microbial environment.

21. The method of claim 20, further comprising adjusting a concentration of nutrients in the microbial environment based at least in part on the terminal electron acceptor utilization rate.

22. The method of claim 21, wherein the terminal electron acceptor utilization rate is indicative of microbial growth rate in the microbial environment.

23. The method of claim 22, further comprising further adjusting the concentration of nutrients based at least in part on at least one of a microbial concentration and flow rate associated with the microbial environment.

24. The method of claim 22, wherein adjusting the concentration of nutrients comprises increasing the concentration of nutrients to increase a microbial growth rate in the microbial environment.
